(19) United States
(12) Patent Application Publication
(10) Pub. No.: US 2002/0099194 A1
(43) Pub. Date: Jul. 25, 2002

(54) Methods to overexpress a foreign gene in a cell or in an animal in vitro and in vivo

(75) Inventors: Argiris Efstratiadis, Englewood, NJ (US); Thomas Ludwig, New York, NY (US); Ana Kljuic, New York, NY (US); Katerina Politi, NYC, NY (US)

Correspondence Address: Cooper and Dunham LLP, 1185 Avenue of the Americas, New York, NY, 10036, US

(73) Assignee: The Trustees of Columbia University, New York, NY

(21) Appl. No.: 09/905,311

(22) Filed: Jul. 13, 2001

Related U.S. Application Data
(60) Provisional application No. 60/218,945, filed on Jul. 14, 2000.

(51) Int. Cl. A01K 67/027; C07H 21/04; C12N 15/00

(52) U.S. Cl. 536/23.2; 435/320.1; 800/14

(57) ABSTRACT

The present invention provides a nucleic acid molecule comprising: (a) a region of DNA which is homologous to a region of an endogenous gene present in a genome of a cell of interest; (b) a first nucleic acid encoding an encephalomyocarditis internal ribosome entry site (EMCV IRES); (c) a second nucleic acid encoding a selectable marker which can be excised from the nucleic acid molecule if the nucleic acid molecule has been integrated into the genome of the cell of interest; and (d) a third nucleic acid encoding a gene of interest. The cell may be an animal cell, a yeast cell or a plant cell. The invention also provides for transgenic non-human animals which are created using the above described construct. The invention also provides methods for making such transgenic animals.

The invention disclosed herein was made with Government support under Grant No. T32CA09503 from U.S. Department of Health and Human Services. Accordingly, the U.S. Government has certain rights in this invention.

### BACKGROUND OF THE INVENTION

Throughout this application, various publications are referenced by author and date. Full citations for these publications may be found listed alphabetically at the end of the specification immediately preceding the claims. The disclosures of these publications in their entireties are hereby incorporated by reference into this application in order to more fully describe the state of the art as known to those skilled therein as of the date of the invention described and claimed herein.

Transgenic animals are animals which bear an exogenous gene (called transgene) in their genome which has been introduced either in them themselves or in an predecessor. Due to the fact that the exogenous gene is also present in the germ cells of these animals, the transgene is transmitted from parent to children so that it is possible to establish lines of transgenic animals from a first founder animal. The introduction of the transgene into the fertilized oocyte maximizes the possibilities of the transgene being present in all the cells, both somatic and germinal, of the founder animal. The latter will transmit the transgene to approximately half of its descendants, which will carry it in all its cells. If the transgene is introduced in a later embryonic stage, the founder animal would be a mosaic since not all its somatic and germinal cells will carry the transgene. This would have the result that a smaller proportion of descendants carries the transgene; however, the descendants which inherit it would carry in all their cells, including the germ cells.

### SUMMARY OF THE INVENTION

The present invention provides a nucleic acid molecule comprising: (a) a region of DNA which is homologous to a region of an endogenous gene present in a genome of a cell of interest; (b) a first nucleic acid encoding an encephalomyocarditis internal ribosome entry site (EMCV IRES); (c) a second nucleic acid encoding a selectable marker which can be excised from the nucleic acid molecule if the nucleic acid molecule has been integrated into the genome of the cell of interest; and (d) a third nucleic acid encoding a gene of interest. The cell may be an animal cell, a plant cell or a yeast cell. The invention also provides for transgenic non-human animals which are created using the above described construct. The invention also provides methods for making such transgenic animals.

### BRIEF DESCRIPTION OF THE FIGURES

FIGS. 1A-1C. FIG. 1A: Genomic organization of the beta-actin locus. FIG. 1B: The targeting vector depicting the regions of homology used and the cassette. FIG. 1C. The beta-actin locus after Cre-mediated recombination. Beta-actin exons are shown in red and lox P sites are shown as green triangles.

FIGS. 2A-1 to 2B-4: Diagrams of the beta actin locus and the targeting vector.

FIG. 3: Targeting of embryonic stem cells using Polyoma virus mT antigen as a gene of interest. DNA from the cells was digested with EcoRI and Pact. The blot was hybridized with a probe external to the targeting vector. The top band represents the wild type non-targeted allele and the bottom band represents the targeted allele. Lanes 2 and 4 show untargeted cells and lanes 1, 3, 5, 6, 7, and 8 show targeted cells.

FIG. 4: Targeted cells after Cre-mediated recombination. a) Targeted cell line upon Cre-mediated recombination. b) Targeted cell line.

FIG. 5: Western blot showing expression of Polyomavirus mT antigen in cells after Cre-mediated recombination. Lanes 1 and 2 represent unrecombined cells as negative controls. Lanes 3-8 represent targeted cells after recombinantion. Lane 9 is a positive control for the antibody.

FIG. 6: Representation of Polyomavirus mT antigen and associated proteins.

FIG. 7: Insertion of the MMTV-myr Akt1-SV40 pA transgene into the M6pr locus. A.) Genomic organization of the M6pr gene. B.) Targeting vector used to knock-n the MMTV-myr Akt1-SV40pA transgene. C.) Predicted structure of the modified M6pr allele. Green boxes indicate exons. The red box indicates the new expression cassette and the blue box corresponds to the transgene.

FIG. 8: The Beta-actin locus and the targeting construct. A) Genomic organization of the B-actin locus used as homology. B) The targeting construct and the locus after homologous recombination.

FIG. 9: The beta-actin locus after cre-mediated recombination.

FIG. 10: Insertion of the MMTV-Shc-SV40 pA transgene into the M6pr locus. A) Genomic organization of the M6pr gene. B) Targeting vector used to knock-in the MMTV-Shc-SV40pA transgene. C) Predicted structure of the modified M6pr allele. Pink boxes indicate exons. The green box indicates the neo expression cassette and the blue box corresponds to the transgene. The colored arrows correspond to approximate transcription start points.

FIG. 11: The beta-actin locus and the targeting construct. A) Genomic organization of the beta-actin locus used as homology B) The targeting construct and the locus after homologous recombination.

FIG. 12: The beta-actin locus after cre-mediated recombination.

FIG. 13: The SV40 Large T Antigen is expressed in all tissues of a mouse that carries the SV40 T Antigen in the beta-actin locus and the HS-cre1* transgene. 100 micrograms of protein extract derived from each tissue of this mouse were immunoprecipitated with an antibody that recognizes the SV40 Large T Antigen (Oncogene Research Products). The immunoprecipitates were loaded onto a 4-15% gradient gel and subjected to SDS-PAGE. The gel was transferred onto a nitrocellulose membrane and immunoblotted with the same antibody. Lane 1: Uterine leiomyosarcoma; Lane 2: Skeltal Muscle; Lane 3: Mammary Gland; Lane 4: Wild-type Liver; Lane 5: Wild-type Embryonic Stem Cells; Lane 6: Embryonic stem cells expression SV40 Large T; Lane 7: Spleen; Lane 8: Pancreas; Lane 9: Lung; Lane 10: Kidney; Lane 11: Heart; Lane 12: Liver; Lane 13: Brain.

### DETAILED DESCRIPTION OF THE INVENTION

The present invention provides a nucleic acid molecule comprising:

(a) a region of DNA which is homologous to a region of an endogenous gene present in a genome of a cell of interest linked to;

(b) a first nucleic acid encoding an encephalomyocarditis internal ribosome entry site (EMCV IRES) linked to;

(c) a second nucleic acid encoding a selectable marker, which can be excised from the nucleic acid molecule if the nucleic acid molecule has been integrated into the genome of the cell of interest, linked to

(d) a third nucleic acid encoding a gene of interest

wherein the first nucleic acid is located immediately following the termination codon of the gene present in the genome of the cell of interest.

The cell may be a yeast cell or any mammalian cell or a plant cell. The beta-actin locus is most preferred as the endogenous gene present in the genome of the cell of interest because the beta-actin promoter causes beta actin to make up about 1% of a cell's protein. The elements listed above are to be constructed in the order give and examples of such constructs can be found in the figures.

One preferred embodiment of the present invention is to use this construct in a cell to overexpress a gene of interest in order to produce a protein of interest in that cell. The invention provides for a method for producing a protein which comprises introducing a nucleic acid as described above into a cell in culture (in vitro) via homologous recombination and culturing the cell under conditions such that the nucleic acid introduced will cause the expression of the gene of interest thereby producing the protein in the cell.

This nucleic acid molecule is a construct which is useful for homologous recombination and for making transgenic animals. The construct has the advantage of allowing the gene of interest to be expressed in a time specific way or in a location specific way in the transgenic animal because the excision of the selectable marker is necessary for expression of the gene of interest. Therefore, if one wishes to have expression of the gene of interest delayed and not expressed initially (i.e. during the embryonic stages of the transgenic animal) one can delay excision of the selectable marker until such time as one wishes to commence expression of the gene of interest.

Tissue specific promoters which drive expression of the recombinase which is used to excise the selectable marker, can be used in conjunction with the nucleic acid molecule described. This tissue specific promoter/recombinase construct would be present at a different location in the genome of the transgenic animal.

In one embodiment of the invention, the animal is a mammal. In another embodiment of the invention, the animal is a sheep, a mouse, a primate, a canine, a feline, a fowl, or a fish. In another embodiment of the invention, the animal is a mouse and the region of DNA of step (a) is homologous to the mouse beta actin gene.

In one embodiment of the invention, the second nucleic acid molecule is flanked by nucleic acid which encodes loxP sites. In using this Cre-lox system, the Cre recombinase is under the control of an inducible promoter or a tissue specific promoter which is also integrated into the genome of the animal of interest. The second nucleic acid may also be flanked by FRT sites (and one can use the flip system of S. cerevisea to excise the selectable marker after the nucleic acid molecule has been integrated into the genome of the animal of interest.

In one embodiment of the invention, the selectable marker of step (c) is a neomyocin resistance gene.

In one embodiment of the invention, the selectable marker of step (c) is any antibiotic resistance gene.

This invention also provides for a method for making a transgenic animal which expresses a foreign gene of interest in a location specific manner in the transgenic animal which comprises stably introducing via homologous recombination the nucleic acid molecule described herein.

The invention also provides for a transgenic non-human animal whose germ or somatic cells contain the nucleic acid molecule which is described above and which was introduced into the mammal, or an ancestor thereof, at an embryonic stage.

In one embodiment of the invention, the non-human animal is a mouse, a sheep, a pig, a dog, a cat, a fowl, a fish, a bovine, or a horse.

The gene of interest may be any gene. The nucleic acid molecule which is the transgene of the transgenic nonhuman mammal may contain an appropriate piece of genomic clone DNA from the mammal designed for homologous recombination.

One major advantage this invention provides is that expression of the gene of interest will be under control of the actin promotor and therefore have very abundant (massive) overexpression.

Transgenic Mice

The methods used for generating transgenic mice are well known to one of skill in the art. For example, one may use the manual entitled “Manipulating the Mouse Embryo” by Brigid Hogan et al. (Ed. Cold Spring Harbor Laboratory) 1986. The transgenic nonhuman mammal may be transfected with a suitable vector which contains an appropriate piece of genomic clone designed for homologous recombination. Alternatively, the transgenic nonhuman mammal may be transfected with a suitable vector which encodes an appropriate ribozyme or antisense molecule. See for example, Leder and Stewart, U.S. Pat. No. 4,736,866 for methods for the production of a transgenic mouse. The disclosures of these publications in their entireties are hereby incorporated by reference into this application in order to more fully describe the state of the art as known to those skilled therein as of the date of the invention described and claimed herein.

This invention also provides for a replicable vector which contains nucleic acid molecule described herein. This expression vector may be a prokaryotic expression vector, a eukaryotic expression vector, a mammalian expression vector, a yeast expression vector, a baculovirus expression vector or an insect expression vector Examples of these vectors include PKK233-2, pEUK-C1, pREP4, pBlueBacHisA, pYES2, PSE280 or pEBVHis. Methods for the utilization of these replicable vectors may be found in Sambrook, et al., 1989 or in Kriegler 1990.

Although there are various possibilities, the most usual manner of introducing the transgene is by microinjection of DNA in the pronucleus of embryos in the single-cell state (Gordon et al., 1980, Proc. Natl. Acad. Sci., U.S.A 77:7380; Brinster et al., 1981, Cell 27:223; Wagner et al., 1981, Proc. Natl. Acad. Sci. U.S.A. 78:6376; Gordon and Ruddle, 1981, Methods Enzymol. 101C:411). Up to the present time, a considerable number of genes have bean introduced and studied in transgenic animals, basically mice (for a survey, see Palmiter and Gordon, 1986, Ann. Rev. Genet. 20:405).

Up to the present time, a considerable number of genes have bean introduced and studied in transgenic animals, basically mice (for a survey, see Palmiter and Gordon, 1986, Ann. Rev. Genet. 20:405). There have also been introduced into transgenic animals recombinant genetic constructions which contain a regulator region and a coding region for a protein which come from different sources. These “compound” transgenes, although present in all the cells of the animal, are only expressed in those tissues which normally activate the specific regulator element used in the genetic construction. In this way, using suitable regulator elements, it is possible to direct the activity of genes of varied interest (clinical, pharmaceutical, biological or biotechnological) to preselected tissues of the transgenic animal. One class of particularly interesting regulator sequences is those which are inducible, due to the fact that they make it possible to regulate the expression of the structural gene to which they are attached, controlling the presence or absence of the inductor required in order to activate said regulator regions.

The generation of transgenic animals is well-established and is known to the corresponding experts (Gorton and Ruddle, 1983, Methods in Enzymol. 101C:1244; Hogan, Constatini and Lacy, 1986, Manipulating a Mouse Embryo. A Laboratory Manual. Cold Spring Harbor Laboratory, Cold Spring Harbor).

One of skill would know how to make transgenic animals. See for example, Methods For Creating Transgenic Animals, U.S. Pat. No. 6,080,912 Bremel , et al. Issued Jun. 27, 2000.

This invention provides for a method for treating a disease caused by a protein deficiency or a lack of a functional protein which comprises administering to a subject suffering from the disease a nucleic acid molecule which encodes the protein wherein the nucleic acid molecule comprises.

(a) a region of DNA which is homologous to a region of an endogenous gene present in a genome of a cell of interest linked to;

(b) a first nucleic acid encoding an encephalomyocarditis internal ribosome entry site (EMCV IRES) linked to;

(c) a second nucleic acid encoding a selectable marker, which can be excised from the nucleic acid molecule if the nucleic acid molecule has been integrated into the genome of the cell of interest, linked to

(d) a third nucleic acid encoding a gene of interest, wherein the nucleic acid molecule is expressed in the subject so as to produce a functional protein within the subject, thereby treating the disease.

In one example, the disease is β-thalassemia or diabetes.

The present invention also provides for a method for determining whether a drug is useful for treating cancer which comprises administering the drug to a transgenic non-human animal which comprises the nucleic acid of claim 1, wherein the gene of interest is an oncogene and the transgenic non-human animal exhibits cancer, which comprises administering the drug to the transgenic non-human animal and determining whether the cancer is ameliorated when compared to an identical transgenic non-human animal which was not administered the drug, thereby determining whether the drug is useful for treating cancer.

This invention is illustrated in the Experimental Details section which follows. These sections are set forth to aid in an understanding of the invention but are not intended to, and should not be construed to, limit in any way the invention as set forth in the claims which follow thereafter.

### EXPERIMENTAL DETAILS

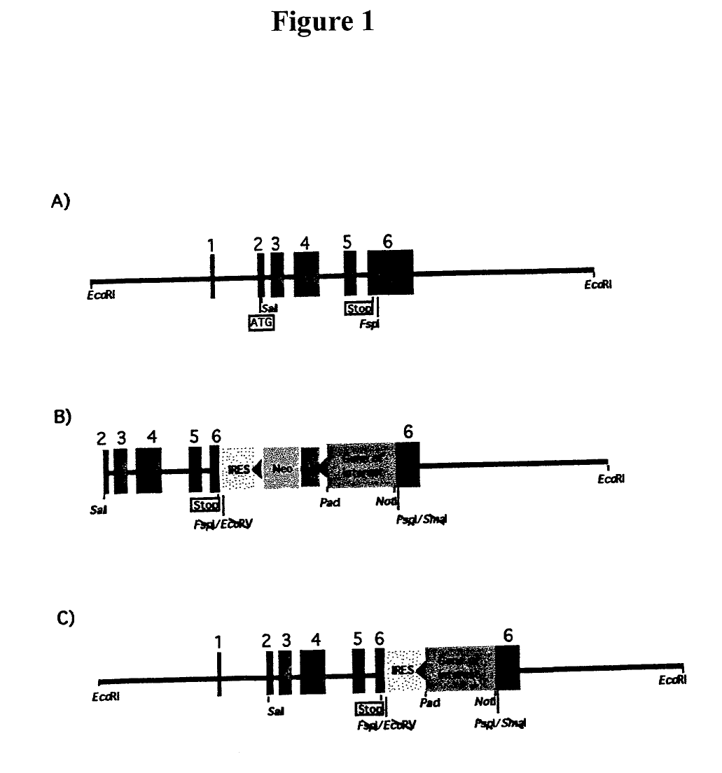

We have developed a method to overexpress any gene with tissue-specifictiy in vitro or in vivo. The method allows any gene to be overexpressed depending on the locus selected to drive expression of the gene. Second, this gene can be overexpressed in a tissue-specific manner. To test this method, we first used mouse embryonic stem cells and subsequently the mouse as a model system. The method we describe is applicable to mammalian cells and organisms but can be extended to any other plant or animal cell or organism provided that an appropriate locus is selected. The method entails a targeting vector composed of a plasmid backbone and genomic DNA homologous to regions of the mouse beta-actin gene interrupted by a cassette. This cassette consists of the Encephalomyocarditis Internal Ribosome Entry Site (EMCV IRES), the neomycin resistance gene flanked by loxP sites and unique cloning sites into which any gene of interest can be inserted (see FIG. 1). When this targeting vector is introduced into mouse cells, it is targeted to the b-actin locus. To avoid disruption of the expression of the endogenous beta-actin gene, the cassette is inserted just downstream of the termination codon of b-actin. The IRES allows CAP-independent translation of a downstream gene. In fact, in the case of the EMCV IRES, the translation initiation complex recognizes the first AUG downstream of the IRES and initiates translation. The neomycin resistance gene followed by the bovine growth hormone polyadenylation signal flanked by lox P sites is inserted as a selectable marker between the IRES and the gene of interest. Therefore, initially, the beta-actin promoter will drive expression of the neomycin gene.

In this way:

1.) we can select targeted colonies of embryonic stem cells or any other mouse cell type after homologous recombination and,

2.) upon Cre-mediated recombination and neomycin excision, the IRES will now be positioned immediately upstream of the gene of interest and will allow it to be translated.

This construct design allows for temporal and tissue specific expression of the gene of interest and is limited only by the specificity of the promoter used to drive the Cre recombinase.

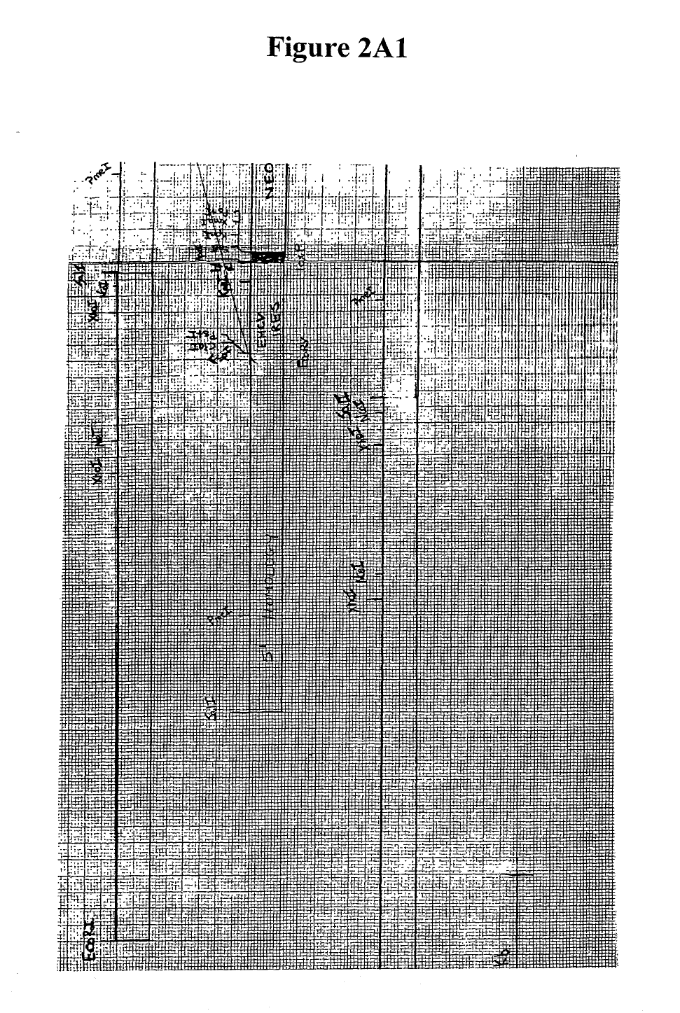

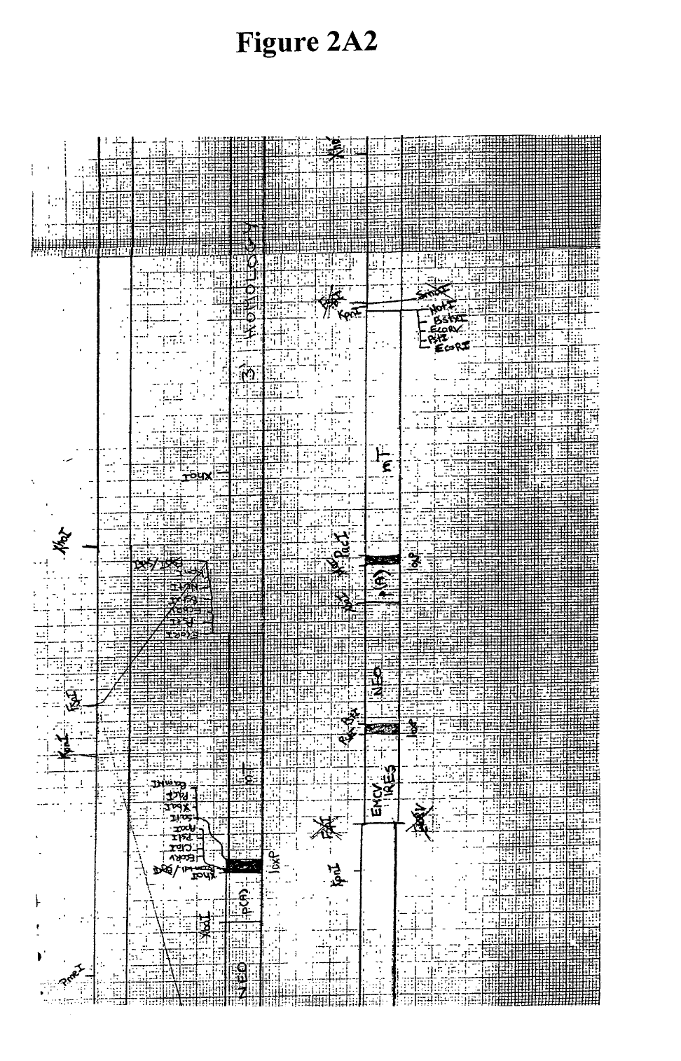

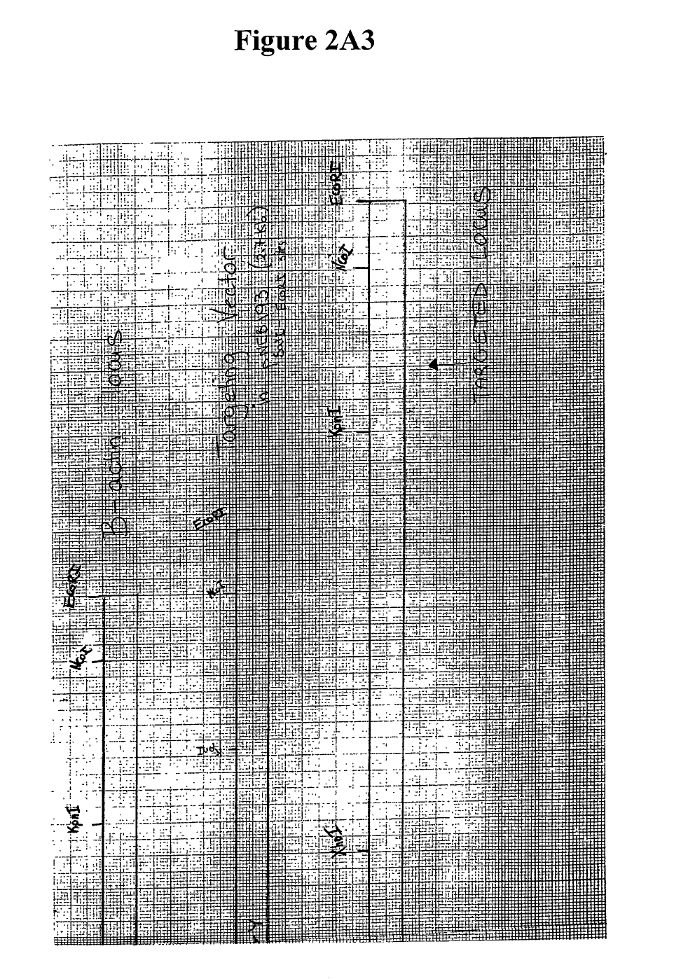

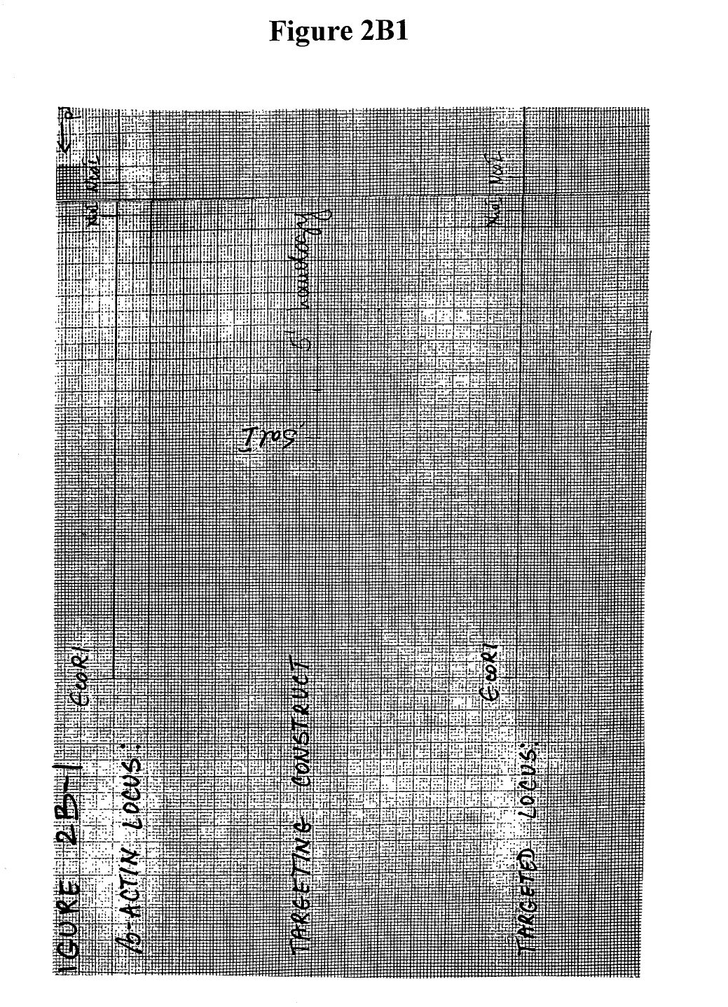

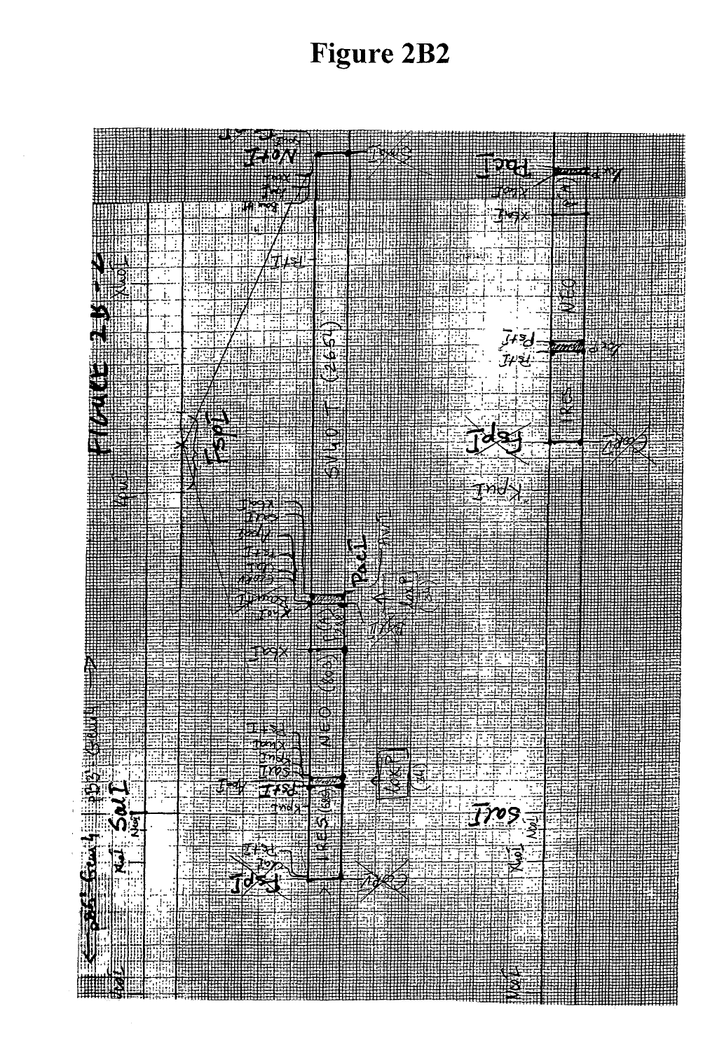

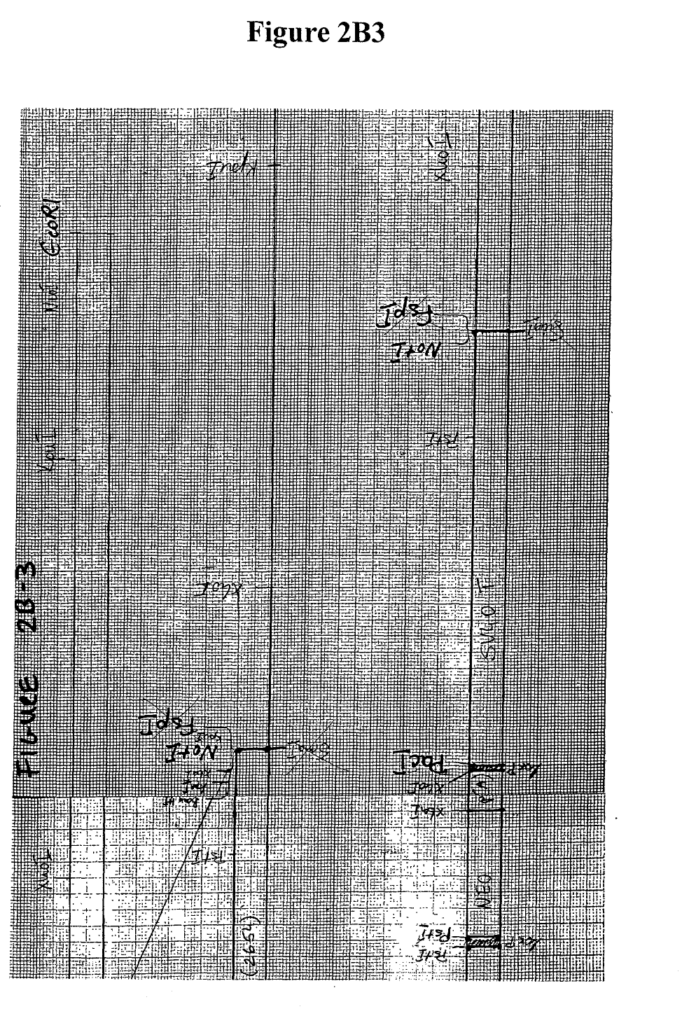

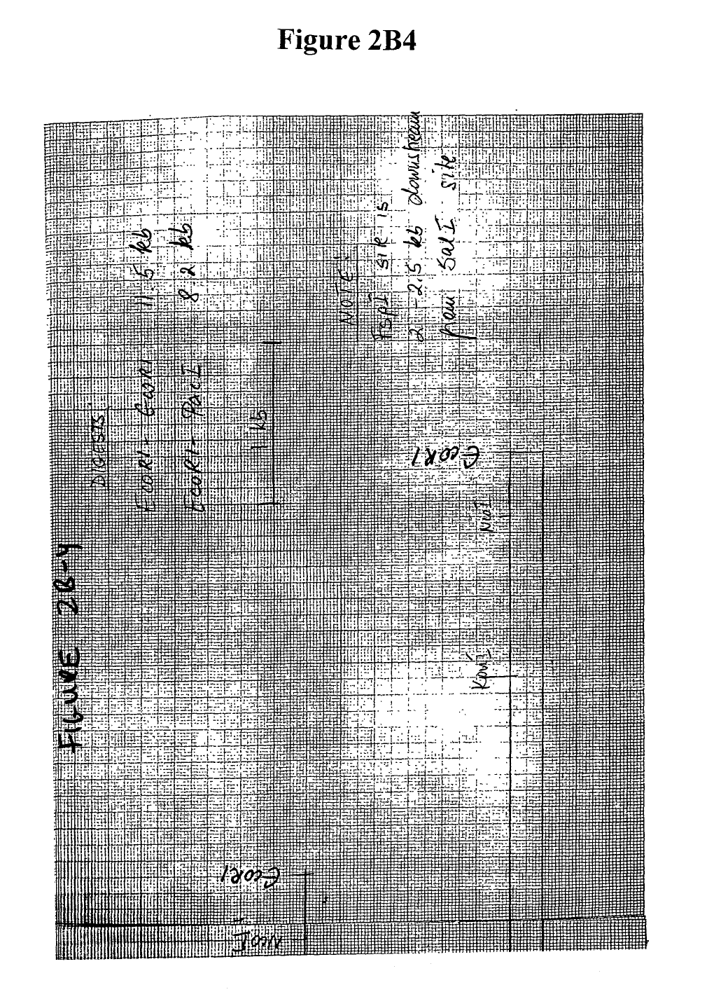

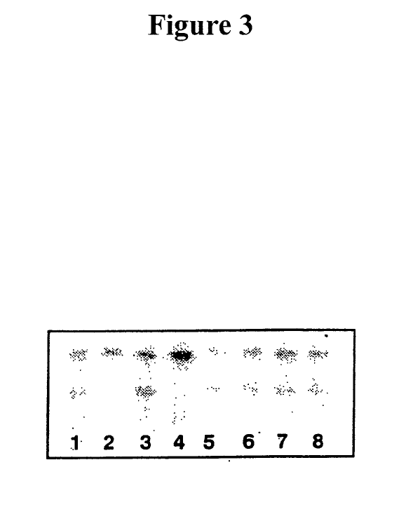

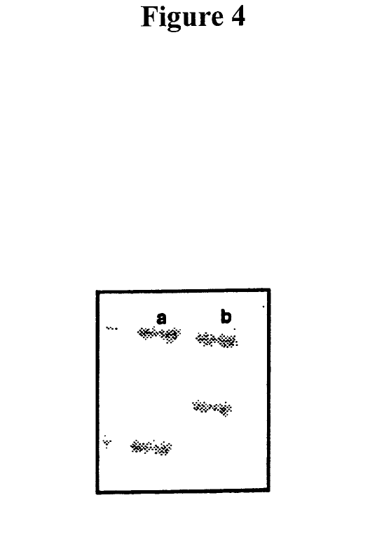

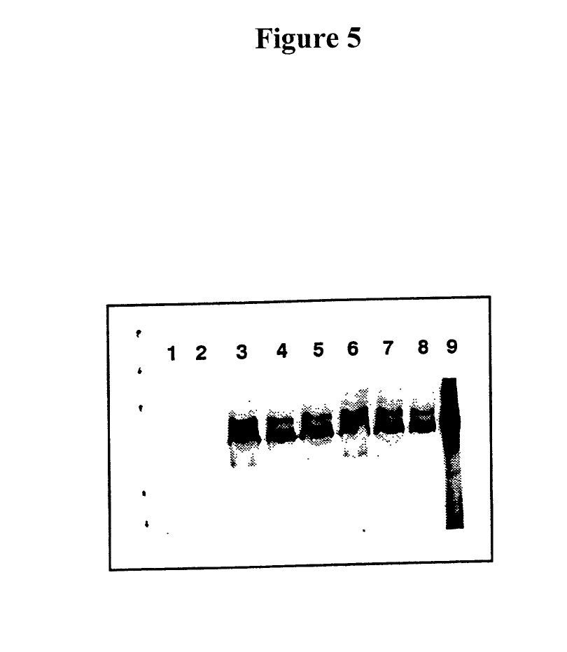

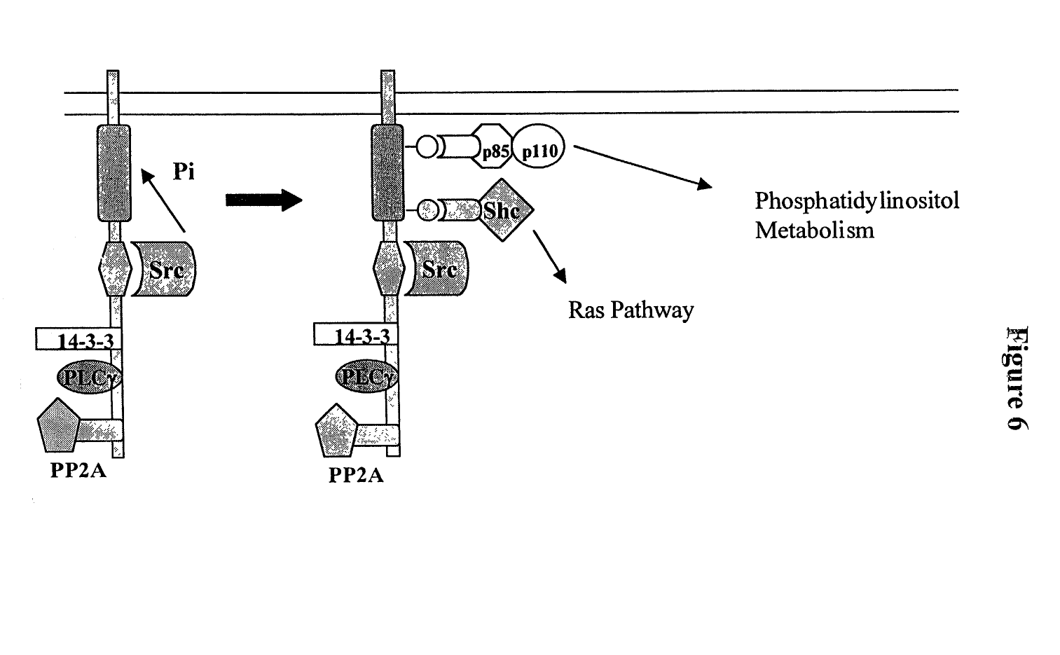

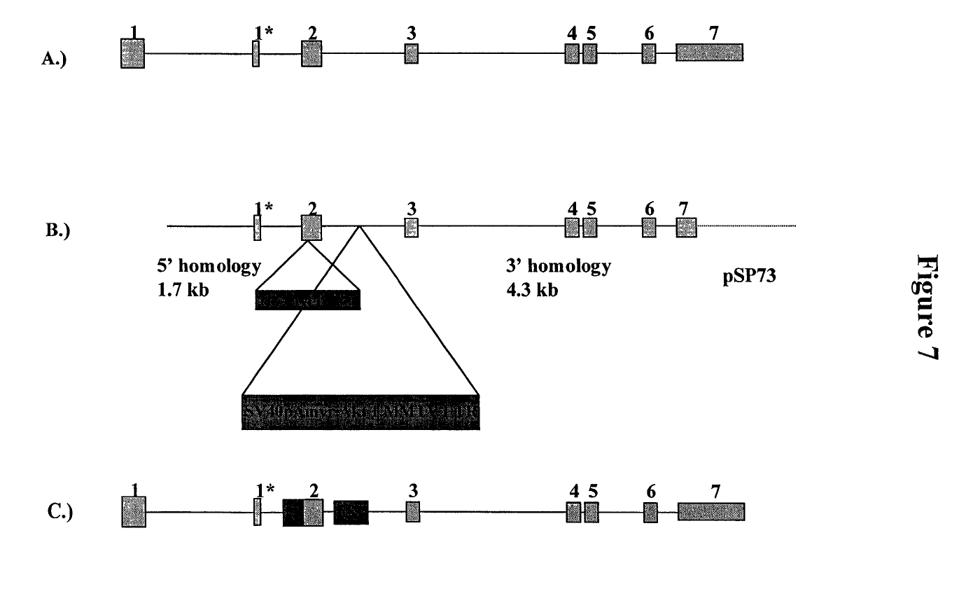

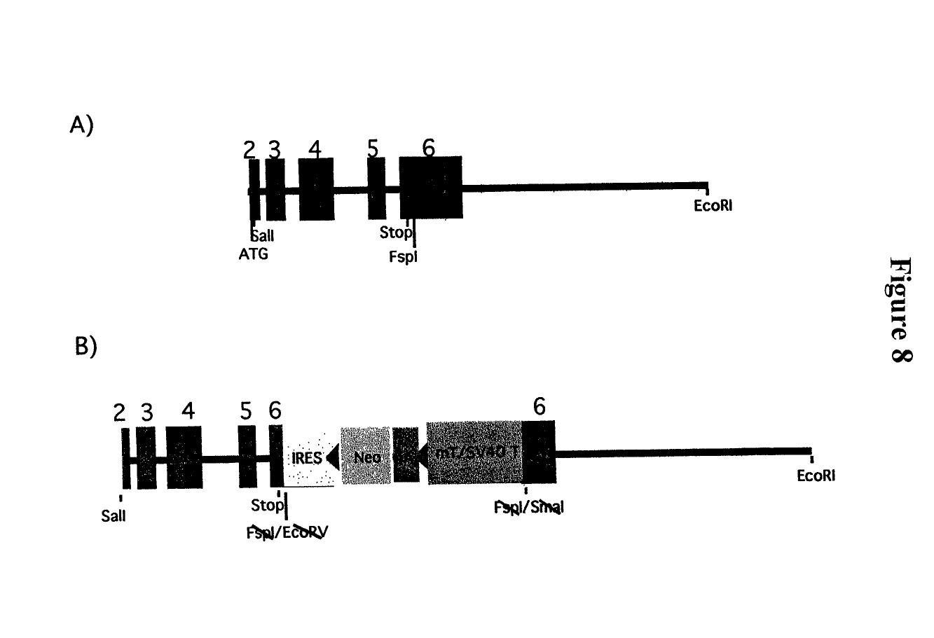

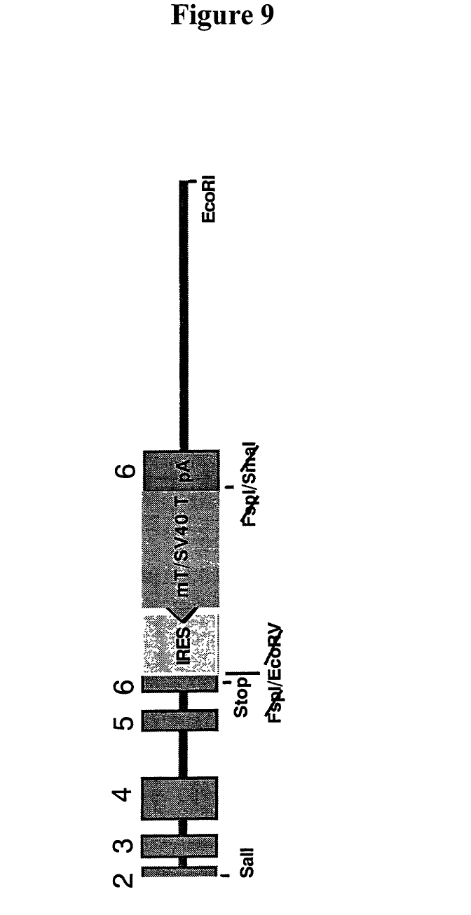

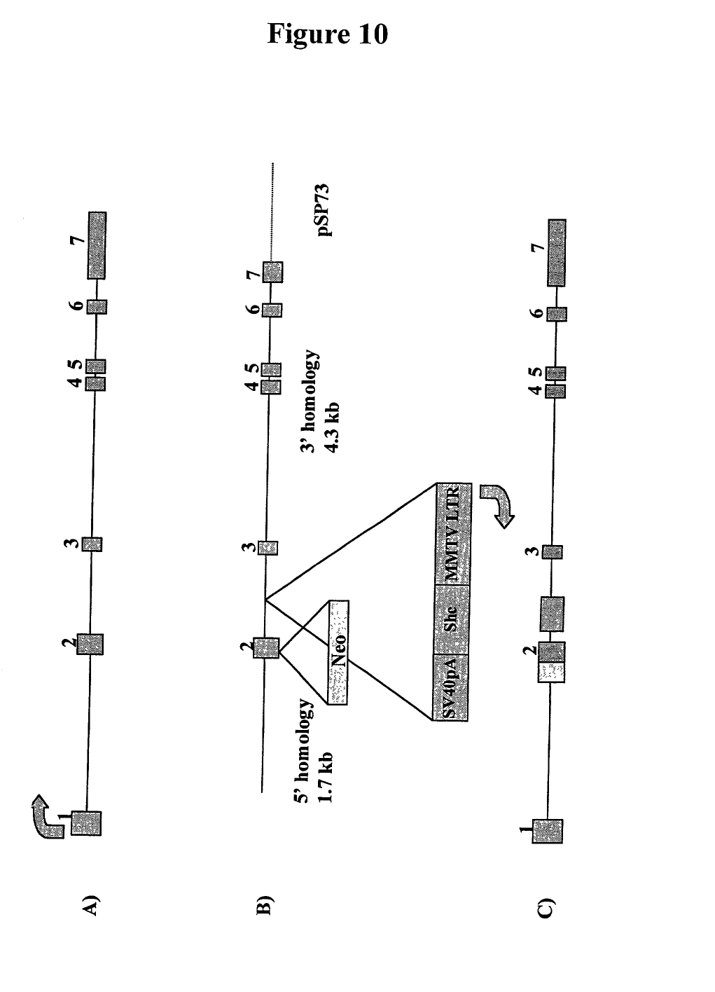

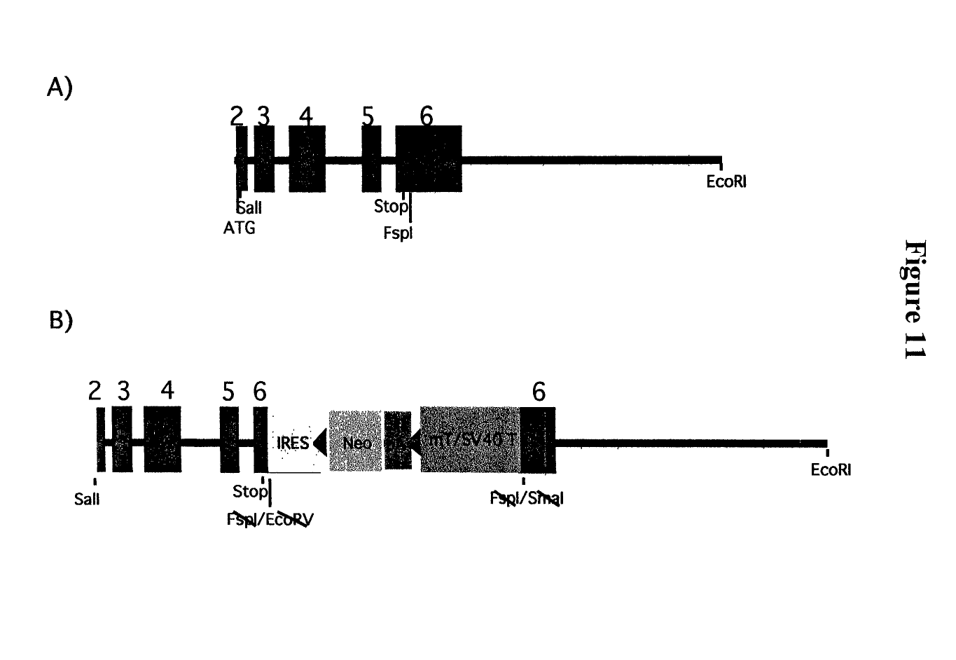

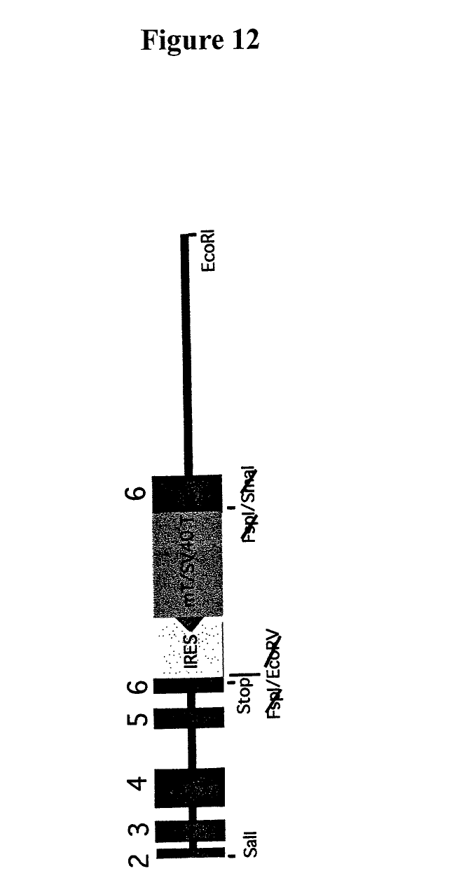

Working maps of constructs are presented as FIGS. 2A and 2B) . The targeting vector for overexpression was completed and electroporated into mouse embryonic stem cells which were chosen as a model to test our system. Properly targeted cells were identified by selecting neomycin resistant cells and analyzing their DNA by Southern blotting. A representative Southern blot is shown in FIG. 3. In order to test our method experimentally we electroporated select targeted cells with Cre recombinase to excise the neomycin resistance gene and therefore position the IRES immediately upstream of the gene to be overexpressed (FIG. 4). To assay for expression of the gene we had inserted into the cassette, we isolated protein from the cells that had undergone correct recombination and analyzed these extracts by Western blotting (FIG. 5).

Our results show that the targeting vector works as expected. We have also inserted three other genes into the targeting vector and have obtained cells with correct targeting and proper recombination demonstrating that any gene can be used for targeting with our targeting vector.

The problems which this invention solves are

1) allows overexpression of any gene in a tissue-specific fashion in vivo or in vitro

2) overcomes the need to generate multiple transgenic lines due to position effects

3) allows different genes to be overexpressed under the control of the same promoter in the same position in the genome and at the same levels.

This invention provides the following advantages:

1. It allows expression at extremely high levels in the cell due to use of the β-actin promoter. β-actin represents 2% of total cellular protein in all cells irrespective of their origin and differentiation state.

2. It allows direct comparison between different genes since the integration site in the genome, copy number and expression levels will be the same since the same promoter is used in its endogenous location in the genome.

3. Insertion of the targeting vector following the termination codon of β-actin ensures that β-actin gene function is not disrupted and thus allows us to use both alleles of β-actin to drive expression of the gene of interest. In this way a gene of interest can be expressed in one or two copies in the genome. In addition, two different genes can be overexpressed from the beta-actin locus in combination in the same cells or organism.

4. It allows production of medically and agriculturally useful products.

5. It allows disruption of pathways important in cancer and other human diseases in cells or organisms.

6. Both positive and negative effectors can be overexpressed using this method.

### Example 1

### Overexpression of Oncogenes in the Mammary Gland Epithelium

It is of interest to study the molecular alterations required for mammary tumorigenesis in the mouse. To approach this problem we have decided to overexpress oncogenes in the mouse mammary gland by generating transgenic mice bearing these oncogenes.

Previous work in this field suggests that the mouse is a good model system for human breast cancer [Cardiff R. D., 1998 #152; Cardiff and Wellings, Seminar at the National Institutes of Health, 1998]. In fact, overexpression of several different oncogenes leads to the development of tumors that are virtually identical to human carcinomas. For example, transgenic mice carrying an activated c-Src oncogene develop scirrhous carcinomas that greatly resemble scirrhous carcinomas in humans (Webster, Cardiff et al. 1995). Of particular interest, increased tyrosine kinase activity attributed to c-Src has been observed in human breast tumors with respect to normal breast tissue (Jacobs and Rubsamen 1983; Rosen, Bolen et al. 1986; Hennipman, van Oirschot et al. 1989; Ottenhoff-Kalff, Rijksen et al. 1992). Also, overexpression of the Neu oncogene in the mouse mammary gland generates nodular comedocarcinomas like those found in humans (Muller, Sinn et al. 1988; Guy, Webster et al. 1992; Guy, Cardiff et al. 1996). Of great importance, comedocarcinomas in humans overexpress the human homologue of Neu, HER-2 (Bartkova, Barnes et al. 1990; Lodato, Maguire et al. 1990; Allred, Clark et al. 1992; Barnes, Bartkova et al. 1992). In addition, expression of the polyomavirus middle T antigen (mT) under control of the mouse mammary tumor virus LTR (MMTV LTR) gives rise to papillary adenocarcinomas very similar to the same tumor type found in humans (Guy, Cardiff et al. 1992).

In contrast to Neu and c-Src, polyomavirus middle T antigen (mT) is not endogenous to the mouse genome. The mT antigen, though, associates with numerous proteins known to be key players in pathways involved in the control of cell proliferation and regulation of cell death, all of which, alone or more probably in combination, could mediate the oncogenic signal from mT [Beck George R. Jr., 1998 #139; Brizuela L., 1994 #151; Dilworth, 1995 #84; Drummond-Barbosa, 1997 #126. These pathways include the Src tyrosine kinase believed to signal through Stat3 and Myc; Shc which signals through Ras; Phosphatidylinositol 3-Kinase (PI3K), which in turn signals through the serine threonine kinase Akt; Protein Phosphatase 2A (PP2A); Phospholipase C g (PLCg) and the protein 14-3-3 (FIG.1). The importance of the Ras pathway and the PI3K pathway in mT-induced tumorigenesis are underscored by an experiment described by Dr. Harold Varmus (Seminar at the Columbia Cancer Center Retreat, October 1999) in which overexpression of mT in glial cells gave rise to the formation of glioblastomas. Glioblastomas were also observed when Akt and Ras were overexpressed in combination in these same cells.

It is important to understand which signaling pathways activated by mT are important in the development of mammary gland tumors. If mT functions in tumorigenesis by activating cellular signaling pathways, overexpression, in the mouse, of components of these same signaling pathways should lead to the development of mammary gland adenocarcinomas like those that develop in mT-expressing mice.

We overexpress members of the different signaling pathways activated by mT in the mammary gland by placing the transgenes under control of the MMTV LTR. In particular, transgenic mice lines are generated expressing either an oncogenic form of PI3K or its constitutively active effector Akt under the control of the MMTV LTR. The transgenes are targeted to a specific locus in the genome in order to directly compare these transgenic animals with MMTV-mT and MMTV-Shc transgenic mice being generated concurrently using the same knock-in approach (FIG. 2).

The MMTV-mT mice developed by Dr. Thomas Ludwig were to serve as a positive control of the procedure used to generate the transgenic mice because it has already been demonstrated that MMTV-mT mice develop mammary adenocarcinomas with a short latency [Guy, 1992 #82]. The MMTV-mT mice generated do not develop mammary adenocarcinomas. In addition, Northern blot analysis indicates that mT is not expressed in these transgenic mice even when the promoter is induced by injecting the mice with dexamethasone.

Transgenic mice were generated carrying a myristylated form of Aktl. These animals do not show any phenotype although the oldest heterozygotes are now 6 months old. MMTV-Shc animals also do not show a phenotype.

In light of these results we have developed an alternative strategy to achieve the same objectives we outlined above.

The alternative strategy is a) to overexpress mT, a potent oncogene that has already been shown to give mammary adenocarcinomas in mice; b) overexpress components of the signaling pathways downstream of mT to determine which of these pathways are important in mouse mammary tumorigenesis by analyzing their phenotypes and comparing them to the phenotype of the mT-expressing mice; c) generate and analyze bi-transgenic mice by crossing mice expressing components of the different signaling pathways that interact with mT.

### Experimental Details

Previously, we had decided that all transgenes need to be driven by the same promoter and inserted into the same genomic site in order for them to be expressed at the same level, allowing accurate comparison of the phenotypes of different mice. In addition, the promoter used should guarantee high levels of expression of the transgene.

We chose to create a construct to target transgenes to the mouse β-actin locus (FIG. 3). Cytoskeletal β-actin represents 2% of total cellular protein therefore using the endogenous β-actin promoter should ensure very high levels of expression of the transgene.

To not disrupt expression of the endogenous β-actin gene, the transgene, preceded by the encephalomyocarditis virus (EMCV) internal ribosome entry site (IRES), is inserted just downstream of the termination codon of β-actin. The IRES allows CAP-independent translation of a downstream gene. In fact, in the case of the EMCV IRES, the translation initiation complex recognizes the first AUG downstream of the IRES and initiates translation.

The neomycin resistance gene followed by the bovine growth hormone polyadenylation signal is flanked by lox P sites and inserted as a selectable marker between the IRES and the transgene (FIG. 3) Therefore, initially, the β-actin promoter will drive expression of the neomycin gene. In this way: 1.) we can select targeted embryonic stem (ES) colonies after homologous recombination and, 2.) upon Cre-mediated recombination and neomycin excision, the IRES will now be positioned immediately upstream of the transgene and will allow it to be translated (FIG. 4) . This construct design allows for temporal and tissue specific expression of the transgene and is limited only by the specificity of the promoter used to drive the Cre recombinase.

Once the construct is ready it is electroporated into ES cells and neomycin resistant colonies will be analyzed for homologous recombination. The construct is then tested in vitro on targeted clones before generating mice. For this purpose, Cre recombinase will be transiently transfected into select targeted ES clones.

Cre should cause excision of the neomycin gene and as a result the transgene can be translated. Expression of the transgene can easily be assayed by Western Blot.

Mice are then generated bearing the construct. Heterozygous mice can be mated to mice that express Cre recombinase under control of the whey acidic protein (WAP) promoter. The female offspring of this cross that carry Cre and our transgene are the experimental animals. Cre under control of the WAP promoter is expressed during pregnancy and lactation specifically in the mammary epithelium ultimately leading to expression of mT in this tissue. The expression pattern of mT is analyzed both by Western blot and immunohistochemistry to ascertain that the transgene is expressed specifically in the mammary epithelium. Furthermore, the animals are observed to determine the incidence and latency of tumor development. The histopathology of any mammary tumors that arise is examined and compared to those of transgenic mice bearing the SV40 T antigen. It is important to determine whether the tumors resemble any human breast carcinomas. To that end, an extensive molecular and biochemical analysis of the tumors is carried out. For example, the levels of mT-associated tyrosine kinase activity is compared between tumor tissue and non-tumor tissue.

Then, substitution of mT with oncogenic versions of the catalytic subunit of PI3K and Akt1. The phenotypes of these mice are analyzed and compared to the phenotype of mammary tumors of mTexpressing mice. Mice are created that express Shc in this locus. Bi-transgenic mice carrying Shc and PI3K or Akt1 are generated and analyzed. Cardiff and his colleagues have underlined the importance, in transgenic mice, of the initiating oncogene in determining the histopathology of the mammary gland tumor [Cardiff and Wellings, Seminar at the National Institutes of Health, 1998]. MMTV-mT mice develop papillary adenocarcinomas, MMTV-Src mice develop scirrhous carcinomas, MMTV-Neu mice develop nodular carcinomas, Ras transgenic mice develop papillary transitional cell carcinomas and Myc transgenic mice develop large cell adenocarcinomas. The histopathology of any tumors that develop in PI3K and Akt transgenic mice are analyzed and compared to tumors arising in mT-expressing mice. If they are the same this could be an indication that activation of the PI3K pathway by mT is crucial in determining the pattern of mT-induced tumors. If they are different, another pathway, or the combination of multiple mT-activated pathways may be more influential in determining the histopathology of the mammary gland tumors.

### Example 2

### Tissue Specific Overexpression of Oncoproteins

Generation of knock-in mice expressing the Shc cDNA under control of the Mouse Mammary Tumor Virus LTR (MMTV LTR) is described. It is important to understand molecular pathways involved in mouse mammary tumorigenessis as a consequence of oncogene activation in this tissue. Polyomavirus middle T (mT) antigen activation in mammary tissue is used as a model pathway for analysis. Polyoma mT has been expressed under control of MMTV LTR; these animals developed multifocal adenocarcinomas with a very short latency and a very high incidence (Guy et al., 1992). Polyoma mT antigen is known to interact with Src tyrosine kinase, PI3 kinase, Shc, PP2A, PLCgamma and the 14-3-3 protein and activate several different cellular pathways (Drummond-Barbosa and DiMaio, 1997). However, not all of these pathways have been implicated in mT induced tumorigenesis. Src tyrosine kinase has been shown to be necessary but not sufficient for tumorigenesis (Guy et al., 1994) (Webster et al., 1995), and both PI3K and Shc have been shown to be required for rapid tumorigenesis (Dahl et al., 1998) (Bronson et al., 1997) (Webster et al., 1998) (Yi et al., 1997). The goal was to generate mono-, bi-, and tri-transgenic animals and compare their tumor formation to the control MMTV-mT expressing mice. In order to compare these animals directly, all of the transgenes were put into the same locus using a “knock-in” approach and therefore ensuring identical levels and patterns of expression. We generated mice expressing Shc under control of MMTV LTR (FIG. 1). The construct was electroporated into 129 SV/eV embryonic stem (ES) cells and clones were identified with proper integration. Two different ES clones were injected into C57BL6 blastocysts and transferred into uteri of pseudo pregnant females. The chimeric offspring were identified by coat color and bred to C57BL6 wild type females in order to generate heterozygous offspring. The heterozygotes, identified by Southern blotting were further bred to homozygosity. As a control experiment for this knock-in approach, MMTV-mT mice were generated, since it is known that mT under control of MMTV LTR gives rise to mammary tumors. Animals at six months of age had not developed any kind of neoplasia. Northern blot analysis was used to look for the expression of mT gene in mammary glands isolated from both heterozygous and homozygous mice. No mT expression was detected. Furthermore, the expression of the transgene was induced by injecting animals with dexamethasone which induces the MMTV promoter. It is possible to have promoter interference if transcription of the transgene is in opposite direction from transcription of the host locus.

It is important to consider whether significant overexpression of the oncoprotein results in rapid mammary tumorigenesis in mice. For these purposes, we decided to examine not only the consequences of mT overexpression, but also the consequences of activation of the early region of the Simian Virus 40 (SV40) in mammary tissue, since mT and SV40 T affect different pathways. The SV40 early region consists of two antigens, large T and small T (referred to as T antigen from here on), which are generated by alternative splicing from one gene. Large T antigen seems to be the driving force in SV40 induced tumorigenesis (Brown, 1986). It has binding sites for several different cellular proteins including p53, pRb, and Hsc-70. SV40 T antigen also performs a variety of functions important to the virus itself. It participates directly in transcription of the viral late genes by interacting with the basal transcription machinery and it encodes ATPase and helicase active regions which are needed for large T involvement in replication (Beck George R. Jr., 1998). However of major interest to us is SV40 T antigen interaction with pRb and p53. These two tumor suppressor genes play key roles in many cellular pathways including cell cycle control, genomic stability and apoptosis (Oren, 1997). An elegant series of experiments in which SV40 T antigen was expressed in the brain choroid plexus or in the lens of the murine eye examined more specifically the roles of each of these tumor supressor genes in tumorigenesis (Fromm L, 1994) (Howes et al., 1994) (Symonds H, 1994) (Pan H, 1994). Using several different transgenic lines expressing the T antigen defective in binding to either pRb or p53 and using intercrosses with pRb and p53 nullyzygous mice it was determined that inactivation of p53 is necessary for inactivation of the apoptotic pathway and inactivation of pRb was necessary for unregulated proliferation. Disruption of both of these pathways led to rapid development of tumors. SV40 T antigen has also been used to generate several different transgenic animals under the control of whey acidic protein (WAP) promoter (Tzeng Yin-jeh, 1993) (Santarelli et al., 1996). WAP is a major mouse milk protein and is expressed in mammary glands during lactation. Its expression is regulated both developmentally and hormonally. Transgenic females in these studies developed mammary adenocarcinomas with high frequency. Both polyomavirus mT antigen and SV40 T antigen have been shown to induce mammary adenocarcinomas with very high frequency when expressed in the mammary tissue. Both are described as potent oncogenes and perturb several different pathways in the cell. However, the pathways that are affected are different. mT acts by mimicking activated growth receptors and activates the MAP kinase pathway as well as the PI3 kinase pathway. On the other hand SV40 T interacts with two tumor suppressor pathways. The common link is the basic need to disrupt regular cell proliferation and control of programmed cell death.

In order to be able to directly compare effects of different transgenes, we firstly had to develop a novel approach because standard transgenic methods were not satisfactory. First, we wanted to overexpress the genes in the same locus in the genome to ensure comparable levels of expression of the two different viral oncoproteins. Second, we needed to find a way to achieve very high levels of expression. To achieve this, we chose to introduce these viral genes into the β-actin locus (FIG. 2). In fact, cytoskeletal β-actin represents 2% of total cellular protein. Therefore, using the β-actin promoter to drive the transgene should ensure very high levels of expression of mT or the SV40 T antigen. In order to avoid disruption of expression at the β-actin locus, we decided to introduce the transgene after the termination codon of β-actin. An Internal Ribosome Entry Site (IRES) was inserted immediately after the termination codon of β-actin. The ribosome should recognize the IRES and initiate cap-independent translation from the first downstream AUG codon. The neomycin resistance gene followed by the bovine growth hormone polyadenylation signal was flanked by lox P sites and inserted as a selectable marker between the IRES and the transgene. Therefore, initially the β-actin promoter will drive expression of the neomycin gene. In this way: (a) targeted ES colonies can be selected and, (b) upon Cre mediated recombination and neomycin excision, the IRES will be positioned immediately upstream of the transgene and will allow its translation (FIG. 3). This construct design allows for temporal and tissue specific expression of the transgene and is limited only by the specificity of the promoter used to drive the cre recombinase. Once the construct is made, it will be electroporated into 129 SV/ev ES cells and proper integration will be determined by Southern blotting. Before using these cells to make transgenic mice, we plan to test for expression of SV40 T antigen in the cells in vitro. The cells will be transiently transfected with Cre recombinase in order to excise neomycin gene and allow IRES to initiate translation of SV40 T antigen. The cells are cotransfected with a different selection marker in order to enrich for Cre recombinase positive cells. Expression of T antigen is be scored by Western blotting. Transgenic mice are made by standard methods previously described. Heterozygous and homozygous animals carrying the transgene are further mated to transgenic mice that express Cre recombinase under control of the WAP promoter. Western blotting is used to determine the expression of T antigen after lactation in mice. The expression is limited to mammary gland. The animals are used further to determine the incidence and latency of tumors. The tumors produced by SV40 T antigen are compared to tumors from animals expressing polyomavirus mT antigen. A comparison of the histopathology of tumors from these two types of transgenic animals will show whether the pathways they affect converge downstream and to which extent. In addition to the analysis of mice, the targeted ES cells can be used for more in vitro analysis of oncogene overexpression. Finally, if this approach proves to be a good tool for direct analysis of different oncogenes, transgenic animals can be made expressing Shc and PI3K initially in order to further dissect signaling pathways in the cell.

### Example 2

### Generation of Mammary Gland Tumors in Polyomavirus mT Antigen Expressing Mice

Mice were generated that carry the Polyomavirus mT antigen in the β-actin locus cassette. The first gene placed in this expression cassette was the polyomavirus mT antigen, which served as a positive control for our strategy because overexpression of mT in the mouse mammary gland is known to give rise to mammary gland adenocarcinomas.

The construct was electroporated into 129SV/eV embryonic stem (ES) cells and neomycin resistant colonies were analyzed for homologous recombination. We tested our construct in vitro on targeted clones by transiently expressing cre in positively targeted ES clones. Expression of cre caused excision of the neomycin gene and as a result mT was expressed as assayed by Western Blot analysis (see FIG. 5). Targeted ES cells were injected into C57B1/6 blastocysts and chimeras derived from these injections were mated with mice that express cre under control of the Whey Acidic Protein (WAP) promoter (Ludwig, T., Fisher, P., Murty, V., and Efstratiadis, A. (2001). Development of mammary adenocarcinomas by tissue-specific knockout of Brca2 in mice. Oncogene 20, 3937-3948). The WAP promoter directs expression of cre to the mammary gland epithelium.

Once mice were generated that carried both mT and cre, we monitored the mice for tumor formation. Presently, we have observed the formation of mammary gland tumors in 100% of mice which carry mT and WAP-cre and which went through at least 1 pregnancy. In these mice, all of the mammary gland tissue is transformed and the tumors appear within 1 month of expression of the Polyomavirus mT antigen. There is data that shows in these mice, mT is expressed exclusively in the mammary gland demonstrating that tissue-specific expression is achieved using this system.

### Example 3

### Generation of Mice that Express the SV40 T Antigen in all Tissues from the 2-cell Stage

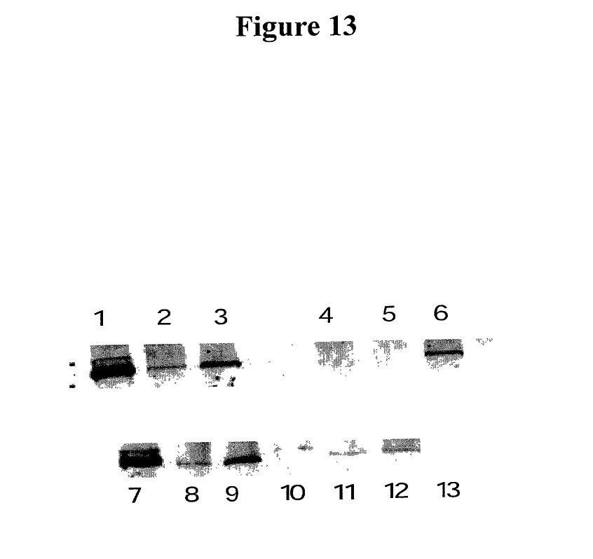

The SV40 T Antigen was introduced into the β-actin expression cassette and generated mice as described for the Polyomavirus Middle T Antigen. The mice carrying the SV40 T Antigen were crossed to mice that carry cre recombinase under control of a heat shock promoter (Dietrich P, Dragatsis I, Xuan S, Zeitlin S, Efstratiadis A. Conditional mutagenesis in mice with heat shock promoter-driven cre transgenes. Mamm Genome. 2000 March; 11 (3) :196-205.; HS-crel*). The HS-crel* mice express cre at the 2-cell stage. Mice carrying both the SV40 T antigen and HS-crel* develop normally and express the SV40 T antigen in all tissues assayed by Western Blot analysis (FIG. 13) demonstrating that this system can be regulated to yield temporal specificity as well as tissue specificity.

### FURTHER APPLICATION OF THE INVENTION

The present invention provides methods to overexpress any gene of interest with temporal specificity and/or tissue-specificity.

As exemplified hereinabove, the Polyomavirus Middle T Antigen was expressed specifically in the mouse mammary gland. This is an example of tissue specific expression of the Polyomavirus Middle T Antigen.

In addition, the SV40 T Antigen was expressed in all mouse tissues beginning at the 2-cell stage of embryonic development. This is an example of temporal specific expression of a gene of interest. The timing of the expression of the SV40 T Antigen coincided with the 2-cell stage of embryonic development of the mouse.

Furthermore, the present invention provides for methods for expression a gene in a stem cell. Both the Polyomavirus Middle T Antigen and the SV40 T Antigen were expressed in mouse embryonic stem cells as exemplified herein.

The present invention provides for methods for treating a disease which is caused by the lack of a protein or the lack of a functional protein which comprises administering to a subject suffering from the disease a nucleic acid molecule which encodes the protein wherein the nucleic acid molecule comprises (a) a region of DNA which is homologous to a region of an endogenous gene present in a genome of a cell of interest linked to; (b) a first nucleic acid encoding an encephalomyocarditis internal ribosome entry site (EMCV IRES) linked to; (c) a second nucleic acid encoding a selectable marker, which can be excised from the nucleic acid molecule if the nucleic acid molecule has been integrated into the genome of the cell of interest, linked to (d) a third nucleic acid encoding a gene of interest, wherein the nucleic acid molecule is expressed in the subject so as to produce a functional protein within the subject, thereby treating the disease.

Using the method described in the immediately preceding paragraph, expression of a gene of interest in an embryonic stem cell or in a specific adult-derived stem cell could be used therapeutically. For example, if one could target the β-globin gene to the β-actin locus in hematopoietic stem cells using this construct it would be possible to cure β-thalassemia.

As another example of the present invention, the insulin gene could be targeted to the β-actin locus and subsequently expressed in pancreatic stem cells to cure diabetes. In this case, the gene of interest is a nucleic acid encoding insulin and the cell of interest is a pancreatic stem cell and the region of DNA homologous to a region of DNA of an endogenous gene present in the genome of the subject, is the β-actin promoter region (the β-actin locus)

Another method provided by the present invention is the generation of transgenic animals for organ transplantation. For example, a gene encoding an antigen/ cell-surface marker can be expressed from the β-actin locus in a transgenic animal whose organs are destined for transplant to humans to prevent the organ from being rejected.

The present invention also provides generation of transgenic animals for drug-screening and/or research purposes. For example, an oncogene can be introduced into the expression cassette which is described herein and and then used to create a transgenic mouse. This mouse can be used to generate tumors in specific tissues susceptible to tumor formation as a result of overexpression of this oncogene. These transgenic mice can be used for screening drugs which would be useful to treat the tumors. This argument is also applicable to genes other than oncogenes.

### REFERENCE

Allred, D. C., G. M. Clark, et al. (1992). “Overexpression of HER-2/neu and its relationship with other prognostic factors change during the progression of in situ to invasive breast cancer.” Hum Pathol 23 (9): 974-9.

Barnes, D. M., J. Bartkova, et al. (1992). “Overexpression of the c-erbB-2 oncoprotein: why does this occur more frequently in ductal carcinoma in situ than in invasive mammary carcinoma and is this of prognostic significance?” Eur J Cancer 28 (2-3): 644-8.

Bartkova, J., D. M. Barnes, et al. (1990). “Immunohistochemical demonstration of c-erbB-2 protein in mammary ductal carcinoma in situ.” Hum Pathol 21 (11): 1164-7.

Guy, C. T., R. D. Cardiff, et al. (1992). “Induction of mammary tumors by expression of polyomavirus middle T oncogene: a transgenic mouse model for metastatic disease.” Mol Cell Biol 12 (3): 954-61.

Guy, C. T., R. D. Cardiff, et al. (1996). “Activated neu induces rapid tumor progression.” J Biol Chem 271 (13): 7673-8.

Guy, C. T., M. A. Webster, et al. (1992). “Expression of the neu protooncogene in the mammary epithelium of transgenic mice induces metastatic disease.” Proc Natl Acad Sci U.S.A. 89 (22): 10578-82.

Hennipman, A., B. A. van Oirschot, et al. (1989). “Tyrosine kinase activity in breast cancer, benign breast disease, and normal breast tissue.” Cancer Res 49 (3): 516-21.

Jacobs, C. and H. Rubsamen (1983). “Expression of pp60c-src protein kinase in adult and fetal human tissue: high activities in some sarcomas and mammary carcinomas.” Cancer Res 43 (4) : 1696-702.

Lodato, R. F., H. C. Maguire, Jr., et al. (1990). “Immunohistochemical evaluation of c-erbB-2 oncogene expression in ductal carcinoma in situ and atypical ductal hyperplasia of the breast.” Mod Pathol 3 (4): 449-54.

Muller, W. J., E. Sinn, et al. (1988). “Single-step induction of mammary adenocarcinoma in transgenic mice bearing the activated c-neu oncogene.” Cell 54 (1): 105-15.

Ottenhoff-Kalff, A. E., G. Rijksen, et al. (1992). “Characterization of protein tyrosine kinases from human breast cancer: involvement of the c-src oncogene product.” Cancer Res 52 (17): 4773-8.

Rosen, N., J. B. Bolen, et al. (1986). “Analysis of pp60c-src protein kinase activity in human tumor cell lines and tissues.” J Biol Chem 261 (29): 13754-9.

Webster, M. A., R. D. Cardiff, et al. (1995). “Induction of mammary epithelial hyperplasias and mammary tumors in transgenic mice expressing a murine mammary tumor virus/activated c-src fusion gene.” Proc Natl Acad Sci U S A 92 (17): 7849-53.

Beck George R. Jr., B. R. Z., and Elizabeth Moran. (1998). Introduction to DNA Tumor Viruses: Adenovirus, Simian Virus 40, and Polyomavirus, D. J. McCance, ed.: American Society for Microbiology).

Bronson, R., Dawe, C., Carroll, J., and Benjamin, T. (1997). Tumor induction by a transformation-defective polyoma virus mutant blocked in signaling through Shc. Proc Natl Acad Sci U S A 94, 7954-8.

Brown, M., M. McCormack, K. G. Zinn, M. P. Farrell, I. Bikel, and D. M. Livingston (1986). A Recombinant Murine Retrovirus for Simian Virus 40 Large T cDNA Transforms mouse fibroblasts to Anchorage-independent growth. Journal of Virology 60, 290-293.

Dahl, J., Jurczak, A., Cheng, L. A., Baker, D. C., and Benjamin, T. L. (1998). Evidence of a role for phosphatidylinositol 3-kinase activation in the blocking of apoptosis by polyomavirus middle T antigen. J Virol 72, 3221-6.

Drummond-Barbosa, D., and DiMaio, D. (1997). Virocrine transformation. Biochim Biophys Acta 1332, M1-17.

Fromm L, S. W., Gunning K, Butel J S, Overbeek PA (1994) . The Retinoblastoma Protein-binding Region of Simian Virus 40 Large T Antigen Alters Cell Cycle Regulation in Lenses of Transgenic Mice. Molecular and Cellular Biology 14, 6743-6754.

Guy, C. T., Cardiff, R. D., and Muller, W. J. (1992). Induction of mammary tumors by expression of polyomavirus middle T oncogene: a transgenic mouse model for metastatic disease. Mol Cell Biol 12, 954-61.

Guy, C. T., Muthuswamy, S. K., Cardiff, R. D., Soriano, P., and Muller, W. J. (1994). Activation of the c-Src tyrosine kinase is required for the induction of mammary tumors in transgenic mice. Genes Dev 8, 23-32.

Howes, K. A., Ransom, N., Papermaster, D. S., Lasudry, J. G., Albert, D. M., and Windle, J. J. (1994). Apoptosis or retinoblastoma: alternative fates of photoreceptors expressing the HPV-16 E7 gene in the presence or absence of p53 [published erratum appears in Genes Dev Jul. 15, 1994; 8 (14):1738]. Genes Dev 8, 1300-10.

Oren, H. R. a. M. (1997). p53; from inductive signal to cellular effect. Current Opinion in Genetics and Development 7, 46-51.

Pan H, G. A. (1994). Altered Cell Cycle Regulation in the Lens of HPV-16 E6 or E7 Transgenic Mice: Implications for Tumor Suppressor Gene Function in Development. Genes and Development 8, 1285-1299.

Santarelli, R., Tzeng, Y. J., Zimmermann, C., Guhl, E., and Graessmann, A. (1996). SV40 T-antigen induces breast cancer formation with a high efficiency in lactating and virgin WAP-SV-T transgenic animals but with a low efficiency in ovariectomized animals. Oncogene 12, 495-505.

Symonds H, K. L., Remington L, Saenz-Robles M, Lowe S, Jacks T, Van Dyke T (1994). p53-dependent Apoptosis Suppresses Tumor Growth and Progression in Vivo. Cell 78, 703-711.

Tzeng Yin-jeh, E. G., Monika Graessmann and Adolf Graessmann (1993). Breast Cancer Formation in Transgenic Animals induced by the Whey Acidic Protein SV40 T Antigen (WAP-SV-T) Hybrid Gene. Oncogene 8, 1965-1971.

Webster, M. A., Cardiff, R. D., and Muller, W. J. (1995). Induction of mammary epithelial hyperplasias and mammary tumors in transgenic mice expressing a murine mammary tumor virus/activated c-src fusion gene. Proc Natl Acad Sci U S A 92, 7849-53.

Webster, M. A., Hutchinson, J. N., Rauh, M. J., Muthuswamy, S. K., Anton, M., Tortorice, C. G., Cardiff, R. D., Graham, F. L., Hassell, J. A., and Muller, W. J. (1998). Requirement for both Shc and phosphatidylinositol 3′ kinase signaling pathways in polyomavirus middle T-mediated mammary tumorigenesis. Mol Cell Biol 18, 2344-59.

Yi, X., Peterson, J., and Freund, R. (1997). Transformation and tumorigenic properties of a mutant polyomavirus containing a middle T antigen defective in Shc binding. J Virol 71, 6279-86.

What is claimed is:

1. A nucleic acid molecule comprising:
(a) a region of DNA which is homologous to a region of an endogenous gene present in a genome of a cell of interest linked to;
(b) a first nucleic acid encoding an encephalomyocarditis internal ribosome entry site (EMCV IRES) linked to;
(c) a second nucleic acid encoding a selectable marker, which can be excised from the nucleic acid molecule if the nucleic acid molecule has been integrated into the genome of the cell of interest, linked to
(d) a third nucleic acid encoding a gene of interest
wherein the first nucleic acid is located immediately following the termination codon of the gene present in the genome of the cell of interest.

2. The nucleic acid of claim 1, wherein the cell of interest in part of an animal.

3. The nucleic acid of claim 2, wherein the animal is a sheep, a mouse, a primate, a canine, a feline, a fowl, or a fish.

4. The nucleic acid of claim 2, wherein the animal is a mouse and the region of DNA of step (a) is homologous to the mouse beta actin gene.

5. The nucleic acid of claim 1, wherein the cell is a yeast cell or a mammalian cell.

6. The nucleic acid of claim 1, wherein the second nucleic acid molecule is flanked by nucleic acid which encodes loxP sites.

7. The nucleic acid of claim 1, wherein the selectable marker of step (c) is a neomyocin resistance gene.

8. The nucleic acid of claim 1, wherein the selectable marker of step (c) is any antibiotic resistance gene.

9. A method for making a transgenic animal which expresses a foreign gene of interest in a location specific manner in the transgenic animal which comprises stably introducing via homologous recombination the nucleic acid molecule of claim 1.

10. A transgenic non-human animal whose germ or somatic cells contain the nucleic acid molecule of claim 1 which was introduced into the mammal, or an ancestor thereof, at an embryonic stage.

11. The transgenic non-human animal of claim 9, wherein the non-human animal is a mouse, a sheep, a pig, a dog, a cat, a fowl, a fish, a bovine, or a horse.

12. A method for treating a disease caused by a protein deficiency or a lack of a functional protein which comprises administering to a subject suffering from the disease a nucleic acid molecule which encodes the protein wherein the nucleic acid molecule comprises
(a) a region of DNA which is homologous to a region of an endogenous gene present in a genome of a cell of interest linked to;
(b) a first nucleic acid encoding an encephalomyocarditis internal ribosome entry site (EMCV IRES) linked to;
(c) a second nucleic acid encoding a selectable marker, which can be excised from the nucleic acid molecule if the nucleic acid molecule has been integrated into the genome of the cell of interest, linked to
(d) a third nucleic acid encoding a gene of interest, wherein the nucleic acid molecule is expressed in the subject so as to produce a functional protein within the subject, thereby treating the disease.

13. The method of claim 12, wherein the disease is β-thalassemia or diabetes.

14. A method for determining whether a drug is useful for treating cancer which comprises administering the drug to a transgenic non-human animal which comprises the nucleic acid of claim 1, wherein the gene of interest is an oncogene and the transgenic non-human animal exhibits cancer, which comprises administering the drug to the transgenic non-human animal and determining whether the cancer is ameliorated when compared to an identical transgenic non-human animal which was not administered the drug, thereby determining whether the drug is useful for treating cancer.
